(19) United States
(12) Patent Application Publication
(10) Pub. No.: US 2004/0078055 A1
(43) Pub. Date: Apr. 22, 2004

(54) Spinal reforming tool

(76) Inventor: Toshihiko Kusumi, Santo-gun (JP)

Correspondence Address: CONNOLLY BOVE LODGE & HUTZ LLP, SUITE 800, 1990 M STREET NW, WASHINGTON, DC, 20036-3425, US

(21) Appl. No.: 10/277,062

(22) Filed: Oct. 22, 2002

(51) Int. Cl. A61B 18/04; A61F 5/00

(52) U.S. Cl. 606/240; 606/32

(57) ABSTRACT

To provide a novel spinal reforming tool that may obtain an extremely high reforming effect only by a simple operation, a pair of reforming projecting portions (3) are provided in parallel with each other on a top portion of a tool body (2) which supports at least an upper half of a human body (1) and with which a back portion of the human body may contact. A reforming groove (4) between the reforming projecting portions (3) is formed in a longitudinal direction of a spine (5) of the back portion of the human body (1) supported on the tool body (2). The reforming projecting portions (3) are slanted in either one of the longitudinal direction, and a drop portion (6) dropping at an abrupt angle is provided at an end portion of the reforming projecting portions (3) on a slanted upward side.

### BACKGROUND OF THE INVENTION

The present invention relates to a spinal reforming tool.

Various spinal reforming tools have been proposed.

### SUMMARY OF THE INVENTION

An object of the present invention is to provide a novel spinal reforming tool by which an extremely high correction effect may be obtained by a simple operation.

According to the present invention, there is provided a spinal reforming tool characterized in that a pair of reforming projecting portions are provided in parallel with each other on a top portion of a tool body which supports at least an upper half of a human body and with which a back portion of the human body may contact, a reforming groove between the reforming projecting portions is formed in a longitudinal direction of a spine of the back portion of the human body supported on the tool body, the reforming projecting portions are slanted in either one of the longitudinal direction, and a drop portion dropping at an abrupt angle is provided at an end portion of the reforming projecting portions on a slanted upward side.

In the spinal reforming tool according to the first aspect of the invention, according to a second aspect of the invention, the human body is supported to the tool body so that a head portion of the human body is located on the slanted upward side of the reforming projecting portions, the spine is caused to be arranged along the reforming groove between the reforming projecting portions, lateral projections of the spine are caused to be depressed against the reforming projecting portions on the slanted uppermost portion before the drop portion when the back portion of the human body is slid on the slanted upward side of the reforming projecting portions so that the back portion falls from the drop portion by its gravitational force, a radial projection of the spine is slid while being clamped in the apex portion reforming groove between the reforming projecting portions of the slanted uppermost portion to thereby perform a twist reform and a projection reform of the spine.

In the spinal reforming tool according to the first aspect of the invention, according to a third aspect of the invention, a joint portion between the reforming projecting portions at the slanted uppermost portion and the drop portion is beveled at a round R.

In the spinal reforming tool according to the second aspect of the invention, according to a fourth aspect of the invention, a joint portion between the reforming projecting portions at the slanted uppermost portion and the drop portion is beveled at a round R.

In the spinal reforming tool according to the first aspect of the invention, according to a fifth aspect of the invention, a drop level of the drop portion from the reforming projecting portions at the slanted uppermost portion is set so that when the head portion or the back portion falls from the drop portion, the gravitational force is likely to work, the spine is applied strongly to the reforming projecting portions by the gravitational force which is applied when the head portion or the back portion of the human body falls from the drop portion to thereby perform a twist reform and a projection reform of the spine.

In the spinal reforming tool according to the second aspect of the invention, according to a sixth aspect of the invention, a drop level of the drop portion from the reforming projecting portions at the slanted uppermost portion is set so that when the head portion or the back portion falls from the drop portion, the gravitational force is likely to work, the spine is applied strongly to the reforming projecting portions by the gravitational force which is applied when the head portion or the back portion of the human body falls from the drop portion to thereby perform a twist reform and a projection reform of the spine.

In the spinal reforming tool according to the fourth aspect of the invention, according to a seventh aspect of the invention, a drop level of the drop portion from the reforming projecting portions at the slanted uppermost portion is set so that when the head portion or the back portion falls from the drop portion, the gravitational force is likely to work, the spine is applied strongly to the reforming projecting portions by the gravitational force which is applied when the head portion or the back portion of the human body falls from the drop portion to thereby perform a twist reform and a projection reform of the spine.

In the spinal reforming tool according to the first aspect of the invention, according to an eighth aspect of the invention, a footrest portion for supporting the lower half of the human body is projectably formed on a bottom portion of the tool body on the slanted downward side of the reforming projecting portions, a heel retainer portion that may support heel portions is provided on a top portion of the footrest portion, the back portion of the human body may be slid on the slanted upward side of the tool body by a force of legs of the human body while the heel portions are retained at the heel retainer portion, and the heel retainer portion is provided in a position where the head portion may fall from the drop portion while curving at least the human body like an arc when the heel portion is retained at the heel retainer portion and the legs are extended.

In the spinal reforming tool according to the sixth aspect of the invention, according to a ninth aspect of the invention, a footrest portion for supporting the lower half of the human body is projectably formed on a bottom portion of the tool body on the slanted downward side of the reforming projecting portions, a heel retainer portion that may support heel portions is provided on a top portion of the footrest portion, the back portion of the human body may be slid on the slanted upward side of the tool body by a force of legs of the human body while the heel portions are retained at the heel retainer portion, and the heel retainer portion is provided in a position where the head portion may fall from the drop portion while curving at least the human body like an arc when the heel portion is retained at the heel retainer portion and the legs are extended.

In the spinal reforming tool according to the seventh aspect of the invention, according to a tenth aspect of the invention, a footrest portion for supporting the lower half of the human body is projectably formed on a bottom portion of the tool body on the slanted downward side of the reforming projecting portions, a heel retainer portion that may support heel portions is provided on a top portion of the footrest portion, the back portion of the human body may be slid on the slanted upward side of the tool body by a force of legs of the human body while the heel portions are retained at the heel retainer portion, and the heel retainer portion is provided in a position where the head portion may fall from the drop portion while curving at least the human body like an arc when the heel portion is retained at the heel retainer portion and the legs are extended.

In the spinal reforming tool according to the tenth aspect of the invention, according to an eleventh aspect of the invention, a support portion for supporting the lateral portion of the back portion of the human body without the displacement the lateral portion in the lateral direction is provided on the lateral direction on the outside of the reforming projecting portions forming the reforming groove.

In the spinal reforming tool according to the first aspect of the invention, according to a twelfth aspect of the invention, a width of the reforming groove is set at a width such that the radial projection existing in the spine and skin of the back portion of the human body may be inserted into the groove.

In the spinal reforming tool according to the ninth aspect of the invention, according to a thirteenth aspect of the invention, a width of the reforming groove is set at a width such that the radial projection existing in the spine and skin of the back portion of the human body may be inserted into the groove.

In the spinal reforming tool according to the eleventh aspect of the invention, according to a fourteenth aspect of the invention, a width of the reforming groove is set at a width such that the radial projection existing in the spine and skin of the back portion of the human body may be inserted into the groove.

A preferred embodiment of the present invention (how to embody the invention) will now be described briefly in view of the effects on the basis of the accompanying drawings.

A back portion of a human body 1 is brought into contact with a tool body 2 and at least an upper half of the human body is supported so that a head portion is located on a slanted upward side of reforming projecting portions 3. A spine 5 is arranged along the reforming groove 4 between the reforming projecting portions 3.

Then, the back portion of the human body 1 is slid on the slanted upward side of the reforming projecting portions 3. When the back portion falls by its gravitational force after the head portion has fallen from a drop portion 6, lateral projections 5B of the spine 5 are brought into pressing contact with the reforming projecting portions 3 at the slanted upper portion located before the drop portion 6 so that a rotational motion is given to the twisted spine back to the normal condition, and a radial projection 5A of the spine 5 that has been strongly depressed by the gravitational force of the human body 1 against the apex portion reforming groove 4A between the reforming projecting portions 3 at the slanted uppermost portion is slid and clamped. Accordingly, the twist reform and the projection reform is applied to the spine 5 that has fallen slidingly through the apex reforming groove 4A.

It is therefore possible to provide a novel spinal reforming tool that is extremely superior in practical use, in which only with a simple operation such that the upper half body is supported so that the spine 5 is simply along the reforming groove 4 on the tool body 2 and the back portion is slidingly moved to fall from the drop portion 6, it is possible to reform the twist or the projection of the spine and to reform the curvature of the spine such as a curved spine or a waist curvature.

Also, for instance, a footrest portion 7 for supporting the lower half of the human body 1 is projectably provided on a bottom portion of the tool body 2 that is on the slanted downward side of the reforming projecting portions 3 and a heel retainer portion 8 for retaining heel portions of feet is provided to a top portion of the footrest portion 7 so that the heel portions are retained at the heel retainer portion 8 and the back portion of the human body 1 may be slid on the slanted upward side of the tool body 2 by a force of legs of the human body. At the same time, if the heel retainer portion 8 is provided in a position where at least the human body 1 is curved like an arc so that the head portion may fall from the drop portion 6 when the heels are retained at the heel retainer portion 8 and the legs are extended, it is possible to provide a spinal reforming tool that is further superior in practical use since the reforming operation may be performed by one-self without any assistance from other persons.

### BRIEF DESCRIPTION OF THE DRAWINGS

FIG. 1 is a perspective view showing an embodiment of the invention.

FIG. 2 is a plan view showing the embodiment of the invention.

FIG. 3 is an illustration showing a condition that an upper half of a human body is supported to a tool body in a use condition and a lower half of the human body is laid on a footrest portion with heels retained at heel retainer portions.

FIG. 4 is an illustration of a positional relationship between a reforming groove and a spine according to the embodiment.

FIG. 5 is an illustration of a condition such that the knees are extended and the human body is curved like an arc from the condition shown in FIG. 3.

FIG. 6 is an illustration of a condition that the back portion is further slid and the head portion is fallen from the drop portion from the condition shown in FIG. 5.

FIG. 7 is an illustration of a condition that the spine is to be reformed according to the embodiment.

FIG. 8 is an illustration of the spine that has been reformed by a spinal reforming device according to this embodiment.

FIG. 9 is an illustration of another use condition according to this embodiment.

### DESCRIPTION OF THE PREFERRED EMBODIMENTS

A specific embodiment of the present invention will now be described.

In this embodiment, a pair of reforming projecting portions 3 are juxtaposed on a top portion of a tool body 2 for supporting at least half an upper human body 1 in contact with a back portion thereof. A reforming groove 4 defined between the reforming projecting portions 3 is formed to extend in a longitudinal direction of a spine 5 of a back portion of the human body 1 supported by the tool body 2.

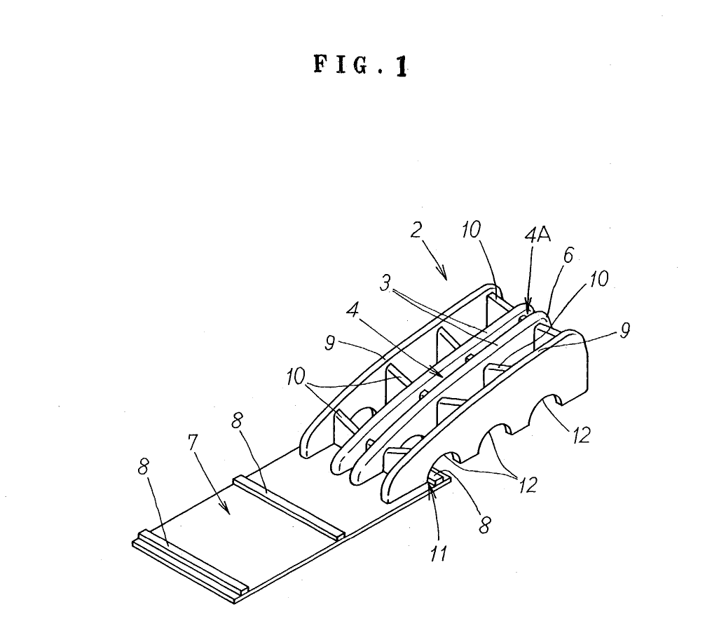

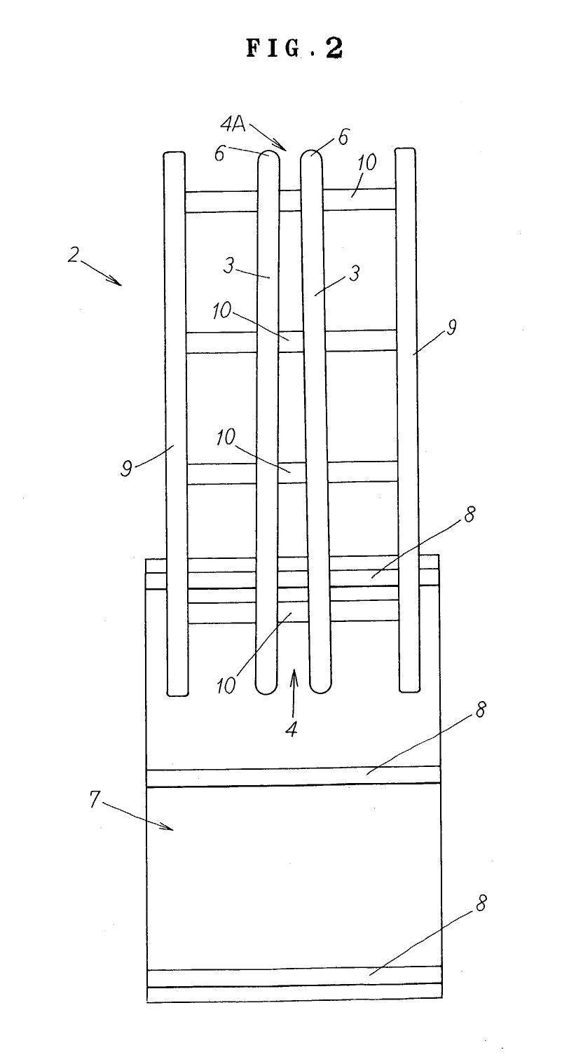

More specifically, as shown in FIGS. 1 and 2, four plate members are held vertically and arranged in parallel with each other at an interval, at the same time, the respective plate members are jointed together by joint plates 10 arranged between the plate members to form the tool body 2 according to this embodiment.

In this embodiment, upper edge portions of two inside ones out of the four plate members forming the tool body 2 are formed into curved edges to form the pair of reforming projecting portions 3 and the space defined between the inside two plate members is formed into the above-described reforming groove 4.

Also, two kinds of inside two plate members whose upper end portions serving as the reforming projecting portions 3 having a thickness of 24 mm and a thickness of 30 mm are prepared. These two kinds of plate members are differently used in accordance with an age of the user or the like.

More specifically, it is known by the applicants' experiments that these two kinds of inside two plate members having the different thickness may enter the human back well to impart correction forces to the spine 5 without any damage such as blood spread due to the break of mussel. The thinner ones have a high reforming force and are suitable for young people whereas the thicker ones have a low reforming force and are suitable for old people.

Also, the outside two plate members serve as support portions 9 for supporting right and left portions of the back portion of the human body 1 to prevent the lateral displacement of the human body 1.

Also, in this embodiment, the reforming projecting portions 3 are slanted on either one of the longitudinal direction, and a drop portion 6 falling at an abrupt angle is formed at an end portion of the reforming projecting portions 3 on the upper side of this slant.

More specifically, the inside two plate members out of the four plate members forming the above-described tool body 2 are made of substantially triangular plate members in which an upper edge portion is slanted upwardly from one side (forward side in FIG. 1) to the other side (far side in FIG. 1) and an abrupt drop closer to a right angle is given at the end portion of the other side of the plate member. Thus, the above-described reforming projecting portion 3 that is the upper end portion of the plate member is slanted so that the end portion of the reforming projecting portion 3 that is on the upper side of the slant may be the above-described drop portion 6.

Also, the joint portion between the slanted top portion of the reforming projecting portion 3 and the drop portion 6 is beveled as a round R. The reason for this is to hardly cause a pain when the back portion slides on and along the joint portion.

Furthermore, two kinds of chamfer like a round 30R which has a high reforming property and a round 50R which has a somewhat weaker reforming property are prepared for the round R of the joint portion between the reforming projecting portion 3 and the drop portion 6 and may be used differently in accordance with the age of the user or the like. Namely, in the same manner as in the thickness of the plate members forming the above-described reforming portions 3, the ones having the high reforming forces are used for young people but ones having low reforming forces and high safety properties should be used for the older people.

Also, in this embodiment, the plate members having substantially the same shape as the inside two plate members are used as the outside two plate members.

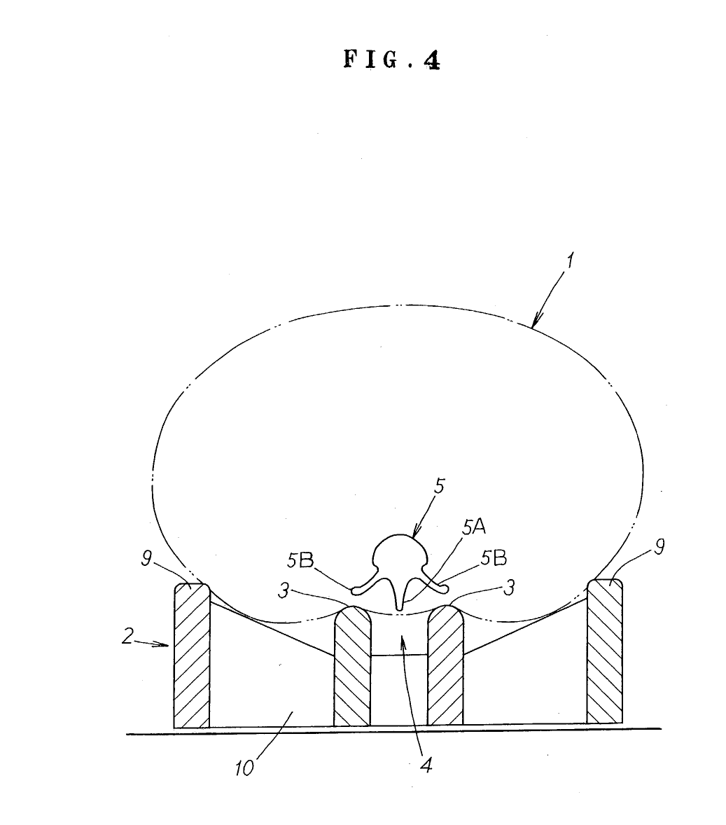

Also, as shown in FIG. 4, in the outside two plate members that are the support portions 9, the upper end portion slanted gently is somewhat projected at a higher level than that of the above-described reforming projecting portion 3 to support stably the right and left portions of the back portion of the human body 1 but is set at a level that is substantially the same or somewhat lower than that of the reforming projecting portion 3 at the end portion on the side of the drop portion 6. It is known by the test results made by the applicants that if such an arrangement is taken, the reforming forces are affected well.

Also, in this embodiment, the drop level of the above-described drop portion 6 from the above-described 5 reforming projecting portion 3 of the slant top portion is set to the drop level at which the gravitational force is likely to work when the head portion or the back portion of the human body 1 falls from the drop portion 6. The spine 5 is strongly depressed against the reforming projecting portion 3 by the gravitational force to be applied when the head portion or the back portion of the human body 1 falls from the drop portion 6 so that the twist reforming and the draw reforming of the spine 5 may be performed by the above-described reforming groove 4.

More specifically, the drop portion 6 is formed so as to have a drop level of about 20 cm from a setup surface of the tool body 2. Namely, in other words, the slant top portion of the reforming projecting portion 3 is formed to have a height of 20 cm from the setup surface.

The height of 20 cm is the optimum height that has been ensured through the actual experiment made by the applicants. If the height is less than 20 cm, the effect of the gravitational force would become insufficient so that the reforming force would become insufficient, whereas if the height is greater than 20 cm, the gravitational force would largely affect dangerously and the tool body 2 per se becomes enlarged, disadvantageously.

Namely, as a result of the setup of the height to 20 cm, it is possible to obtain the extremely excellent reforming effect of the spine 5.

Also, in this embodiment, the width of the above-described reforming groove 4 is set to a level such that radial projections 5A existing in the spine and skin of the back portion of the human body 1 may be inserted into the groove.

More specifically, an interval of the respective apex portions of the above-described pair of reforming projecting portions 3 is set at 48 to 50 mm, and an interval of the confronting inner side surfaces of the pair of reforming projecting portions 3 is set at 24 mm.

The width of the reforming groove 4 is also obtained as a result of the actual experiments made by the applicants.

Also, a depth of the reforming groove 4 is set at 24 mm. It is confirmed by the test results obtained by the applicants that if the depth is set at this level, even if the wear of the user has a thickness to some extent, the radial projections 5A of the spine 5 is likely to enter the reforming groove 4.

Also, in this embodiment, the pair of reforming projecting portions 3 are provided so that the groove width of the reforming groove 4 is gradually expanded downwardly as shown in FIG. 2. Accordingly, even if the tool is applied to a person whose spine 5 is deformed at some portion, there is no fear that the spine 5 projects outside of the pair of reforming projecting portions 3, and the spine 5 may be arranged between the above-described reforming projecting portions 3 so that the curve of the spine 5 may be reformed by the sliding movement of the back portion as the groove width is decreased.

Also, in this embodiment, a lateral width of the above-described tool body 2 (i.e., an interval between the outer two plate members) is set at about 30 cm. If the lateral width of the tool body 2 is too large or too small, the lateral stability of the tool body is lost. It is confirmed by test results obtained by the applicant that the lateral width of about 30 cm is the best for a Japanese person whose statue is about 170 cm.

Also, a length of the tool body 2 is set at about 77 cm that is at a length such that a portion from a rear head portion to a hip bone of a general adult may lie thereon. Incidentally, if the length of the tool body 2 (i.e., the length of the reforming projecting portions 3) is too short, an area where the tool body is to be brought into contact with the back portion of the human body is small, which is dangerous so that the projecting portions are likely to bite into the human back and the sliding movement of the back portion is not smoothly taken. However, it is confirmed by the test results obtained by the applicants that, if the length is set at this length, the reforming force by the suitable bite into the back portion and the smooth sliding movement of the back portion may be obtained.

Also, in this embodiment, a footrest portion 7 for supporting a lower half portion of the human body 1 is projectingly provided on a bottom portion of the above-described tool body 2 on the lower slanted portion of the above-described reforming projecting portion 3, and a heel retainer portion 8 for supporting heel portions of feet is provided on the top portion of the footrest portion 7 so that the back portion of the human body 1 may slidingly move on the slanted upward side of the above-described tool body 2 by a force of legs of the human body.

More specifically, in this embodiment, a rectangular plate member that has substantially the same size as that of the tool body 2 is used as the above-described footrest portion 7. The latter may be adapted to detachably mounted on the bottom portion of the tool body 2.

Also, a plurality of lateral rods are provided on a top surface of the footrest 7 and are used as the above-described heel retainer portion 8.

Also, explaining the detachable mechanism between the footrest portion 7 and the tool body 2, in this embodiment, the footrest portion 7 and the tool body 2 are coupled with each other through a positioning means 11 for coupling the two components in the positioning condition.

Explaining the positioning means 11, a plurality of cutaway retainer windows 12 are formed on the bottom portion of the tool body 2, and the cutaway retainer window 12 is retained at the above-described heel retainer portion 8 so that the footrest portion 7 may be mounted on the tool body 2 under the positioning condition. Namely, the heel retainer portion 8 has the two functions for retaining the footrest portion 7 to the tool body 2 and for retaining the heels as expressed literally.

Also, the heel retainer portions 8 and the cutaway retainer windows 12 are both provided in a plurality of positions so that the projecting position of the footrest portion 7 to the tool body 2 may be changed in conformity with the statue of the user or the like and furthermore in no use, the footrest portion 7 may be received just under the tool body 2 to thereby save the space therefor.

Also, in this embodiment, the heel retainer portion 8 is provided in a position where at least the human body 1 may be curved like an arc with the head falls from the above-described drop portion 6 when the user extends his or her legs with his or her heels retained at the heel retainer portion 8.

The specific method of using the spinal reforming tool according to this embodiment will now be described.

First of all, the thickness of the plate members forming the reforming projecting portions 3 and the tool having the suitable round R of the coupling portion with the reforming projecting portions 3 just before the drop portion 6 are selected in conformity with the age of the user.

As shown in FIGS. 1 and 2, the cutaway retainer window 12 of the bottom portion of the tool body 2 is retained at a desired heel retainer portion 8 of the footrest portion 7 provided on the setup surface to thereby set the tool body 2.

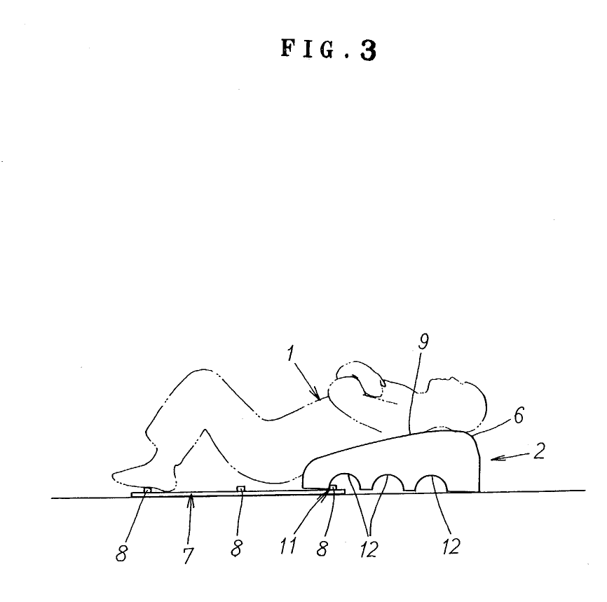

As shown in FIG. 3, the lower half human body lies on the footrest portion 7 retaining his or her heel at the heel retainer portion 8 and bending his or her knees so that the upper half human body lies on the tool body 2 with his or her head located on the slanted upward side of the reforming projecting portions 3 and with the back portion of the human body 1 in contact with the tool body 2. Thus, as shown in FIG. 4, the spine 5 is along the above-described reforming groove 4 between the reforming projecting portions 3.

Both arms of the user are cross with each other on his or her breast with the confirmation as to whether the rear head portion is located in the intermediate portion of the reforming projecting portion 3.

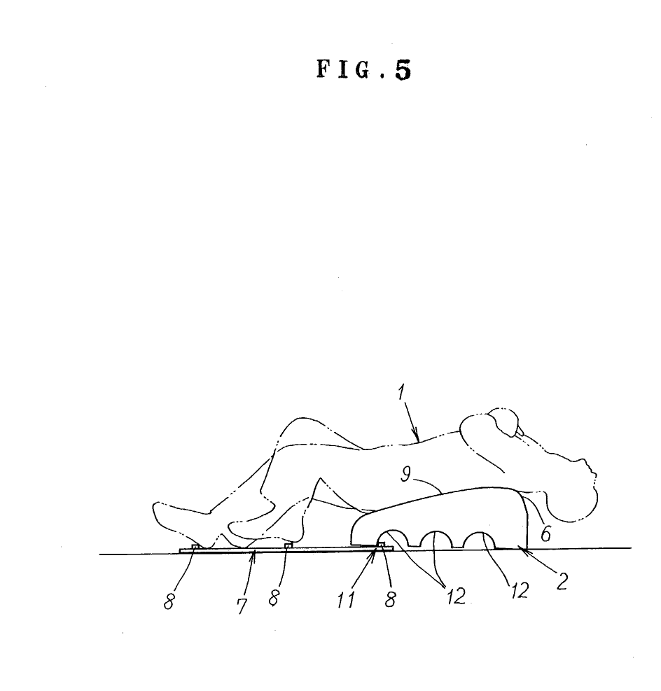

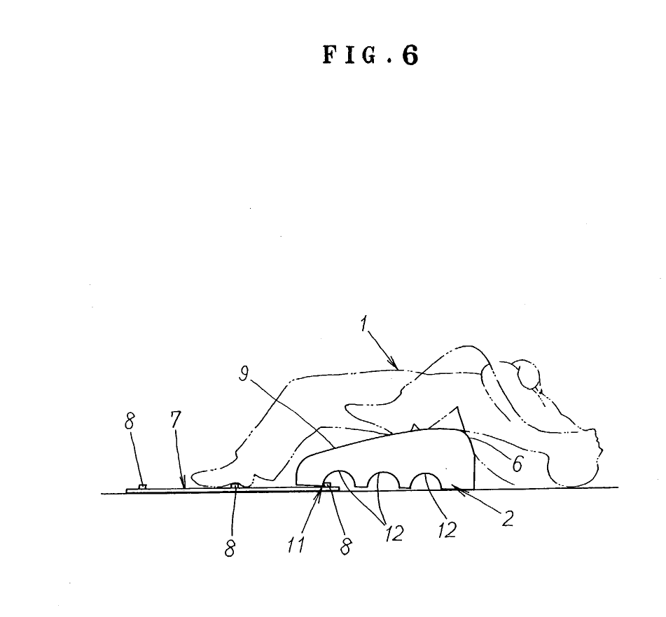

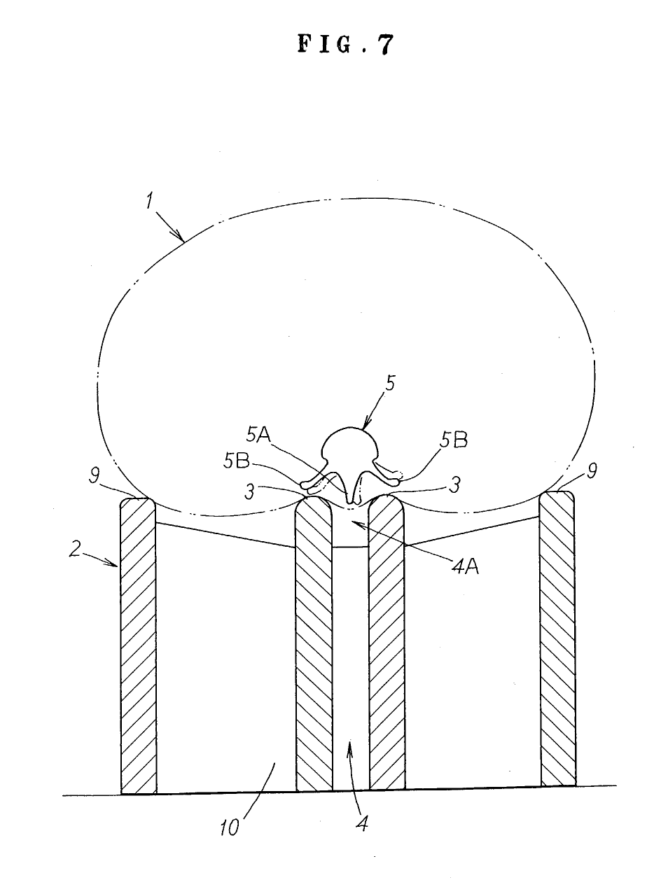

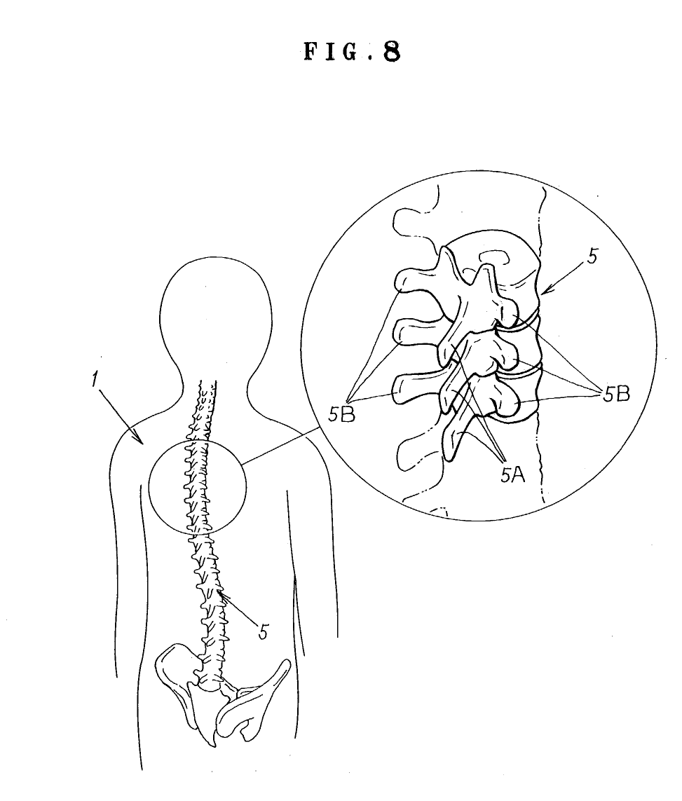

Then, the user extends his or her knees so that the back portion of the human body 1 slidingly moves on the slanted upward side of the reforming projecting portions 3 (see FIG. 5), and the back portion falls due to the gravitational force subsequently with the head portion dropping from the drop portion 6 (see FIG. 6). The lateral projections 5B of the spine 5 are brought into contact with the above-described reforming projecting portion 3 of the slanted uppermost portion just before the drop portion 6 so that a rotational motion is applied to the twisted spine back to the normal condition and the radial projections 5A of the spine 5 strongly applied to the apex portion reforming groove 4A between the reforming projecting portions 3 of the slanted uppermost portion due to a weight of the human body 1 and a gravitational force are clamped and slidingly moved (see FIG. 7). Thus, the spine 5 passes along and in the apex portion reforming groove 4A to reform the twist and the projections one after one so that the spine 5 may be reformed as shown in FIG. 8.

The motion for sliding the back portion along the upper portion of the tool body 2 is performed for about two seconds. If this motion is performed three times to five times a day, the effect is ensured. Also, this motion is continuously performed, it is recommendable that the user bends his or her elbows and knees back to the original position (the condition shown in FIG. 3) while entering and gripping the above-described cutaway retainer window 12 by his or her both hands under the condition that the back portion drops on the setup surface. Namely, the cutaway retainer window 12 is adapted to have a function as the handrails for this purpose.

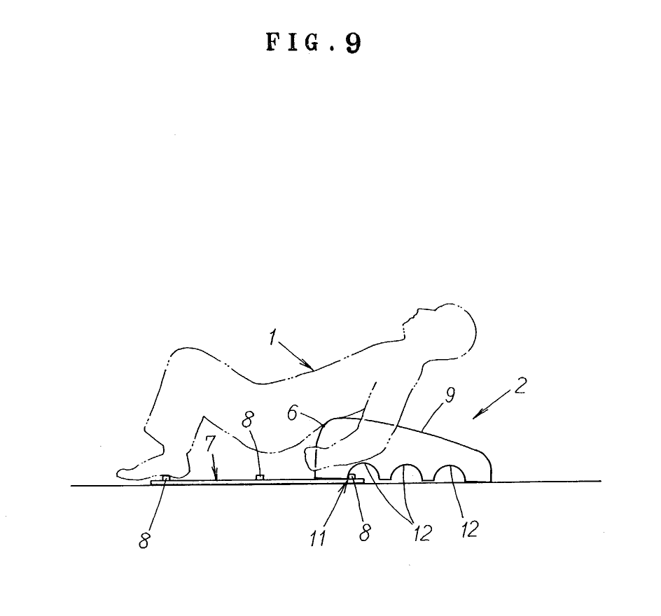

Also, FIG. 9 shows a state where the tool body 2 according to this embodiment is used in the revere direction as an example in a different use.

More specifically, first of all, the waist is applied to the drop portion 6, and the user bends his or her knees and sits on the hoot rest portion 7. The heels are retained at the heel retainer portion 8 and the hands grip the end portions of the outside two plate members that are the support portions 9.

Subsequently, the knees are expended to expand the breast outward.

This method of use is effective to a reforming of the waist bone and the hip bone.

Incidentally, it is apparent that the present invention is not limited to the specific embodiment but may be suitably changed or modified for any other specific structure of respective constituents.

With the above-described structure according to the present invention, it is possible to provide the novel spinal reforming tool that is extremely superior in practical use, in which only with the simple operation such that the upper half body is supported so that the spine is simply along the reforming groove on the tool body and the back portion is slidingly moved on the slanted upward side to fall from the drop portion, it is possible to reform the twist or the projection of the spine and to reform the curvature of the spine such as a curved spine or a waist curvature.

Also, according to the second aspect of the invention, it is possible to provide the novel spinal reforming tool that is extremely superior in practical use, in which the above-described resultant effect is exhibited without fail and the reforming of the spine is extremely well performed.

Also, according to the third and fourth aspects of the invention, it is possible to provide the novel spinal reforming tool that is further superior in practical use, in which there is no remarkable pain when the back portion of the human body falls slidingly along the drop portion.

Also, according to the fifth to seventh aspects of the invention, it is possible to provide the novel spinal reforming tool that is further superior in practical use, in which the reforming of the spine is extremely well performed.

Also, according to the eighth to tenth aspects of the invention, it is possible to provide the novel spinal reforming tool that is further superior in practical use, in which the above-described reforming operation may be performed by the user himself or herself without any help from other people.

Also, according to the eleventh aspect of the invention, it is possible to provide the novel spinal reforming tool that is further superior in practical use, in which the back portion of the human body may be slidingly displaced on the body under the condition that the lateral displacement hardly occurs.

Also, according to the twelfth to fourteenth aspects of the invention, it is possible to provide the novel spinal reforming tool that is further superior in practical use, in which the reforming of the spine is extremely well performed.

What is claimed is:

1. A spinal reforming tool characterized in that a pair of reforming projecting portions are provided in parallel with each other on a top portion of a tool body which supports at least an upper half of a human body and with which a back portion of the human body may contact, a reforming groove between the reforming projecting portions is formed in a longitudinal direction of a spine of the back portion of the human body supported on the tool body, the reforming projecting portions are slanted in either one of the longitudinal direction, and a drop portion dropping at an abrupt angle is provided at an end portion of the reforming projecting portions on a slanted upward side.

2. The spinal reforming tool according to claim 1, characterized in that the human body is supported to the tool body so that a head portion of the human body is located on the slanted upward side of the reforming projecting portions, the spine is caused to be arranged along the reforming groove between the reforming projecting portions, lateral projections of the spine are caused to be depressed against the reforming projecting portions on the slanted uppermost portion before the drop portion when the back portion of the human body is slid on the slanted upward side of the reforming projecting portions so that the back portion falls from the drop portion by its gravitational force, a radial projection of the spine is slid while being clamped in the apex portion reforming groove between the reforming projecting portions of the slanted uppermost portion to thereby perform a twist reform and a projection reform of the spine.

3. The spinal reforming tool according to claim 1, characterized in that a joint portion between the reforming projecting portions at the slanted uppermost portion and the drop portion is beveled at a round R.

4. The spinal reforming tool according to claim 2, characterized in that a joint portion between the reforming projecting portions at the slanted uppermost portion and the drop portion is beveled at a round R.

5. The spinal reforming tool according to claim 1, characterized in that a drop level of the drop portion from the reforming projecting portions at the slanted uppermost portion is set so that when the head portion or the back portion falls from the drop portion, the gravitational force is likely to work, the spine is applied strongly to the reforming projecting portions by the gravitational force which is applied when the head portion or the back portion of the human body falls from the drop portion to thereby perform a twist reform and a projection reform of the spine.

6. The spinal reforming tool according to claim 2, characterized in that a drop level of the drop portion from the reforming projecting portions at the slanted uppermost portion is set so that when the head portion or the back portion falls from the drop portion, the gravitational force is likely to work, the spine is applied strongly to the reforming projecting portions by the gravitational force which is applied when the head portion or the back portion of the human body falls from the drop portion to thereby perform a twist reform and a projection reform of the spine.

7. The spinal reforming tool according to claim 4, characterized in that a drop level of the drop portion from the reforming projecting portions at the slanted uppermost portion is set so that when the head portion or the back portion falls from the drop portion, the gravitational force is likely to work, the spine is applied strongly to the reforming projecting portions by the gravitational force which is applied when the head portion or the back portion of the human body falls from the drop portion to thereby perform a twist reform and a projection reform of the spine.

8. The spinal reforming tool according to claim 1, characterized in that a footrest portion for supporting the lower half of the human body is projectably formed on a bottom portion of the tool body on the slanted downward side of the reforming projecting portions, a heel retainer portion that may support heel portions is provided on a top portion of the footrest portion, the back portion of the human body may be slid on the slanted upward side of the tool body by a force of legs of the human body while the heel portions are retained at the heel retainer portion, and the heel retainer portion is provided in a position where the head portion may fall from the drop portion while curving at least the human body like an arc when the heel portion is retained at the heel retainer portion and the legs are extended.

9. The spinal reforming tool according to claim 6, characterized in that a footrest portion for supporting the lower half of the human body is projectably formed on a bottom portion of the tool body on the slanted downward side of the reforming projecting portions, a heel retainer portion that may support heel portions is provided on a top portion of the footrest portion, the back portion of the human body may be slid on the slanted upward side of the tool body by a force of legs of the human body while the heel portions are retained at the heel retainer portion, and the heel retainer portion is provided in a position where the head portion may fall from the drop portion while curving at least the human body like an arc when the heel portion is retained at the heel retainer portion and the legs are extended.

10. The spinal reforming tool according to claim 7, characterized in that a footrest portion for supporting the lower half of the human body is projectably formed on a bottom portion of the tool body on the slanted downward side of the reforming projecting portions, a heel retainer portion that may support heel portions is provided on a top portion of the footrest portion, the back portion of the human body may be slid on the slanted upward side of the tool body by a force of legs of the human body while the heel portions are retained at the heel retainer portion, and the heel retainer portion is provided in a position where the head portion may fall from the drop portion while curving at least the human body like an arc when the heel portion is retained at the heel retainer portion and the legs are extended.

11. The spinal reforming tool according to claim 10, characterized in that a support portion for supporting the lateral portion of the back portion of the human body without the displacement of the lateral portion in the lateral direction is provided on the lateral direction on the outside of the reforming projecting portions forming the reforming groove.

12. The spinal reforming tool according to claim 1, characterized in that a width of the reforming groove is set at a width such that the radial projection existing in the spine and skin of the back portion of the human body may be inserted into the groove.

13. The spinal reforming tool according to claim 9, characterized in that a width of the reforming groove is set at a width such that the radial projection existing in the spine and skin of the back portion of the human body may be inserted into the groove.

14. The spinal reforming tool according to claim 11, characterized in that a width of the reforming groove is set at a width such that the radial projection existing in the spine and skin of the back portion of the human body may be inserted into the groove.
